(19) United States
(12) Patent Application Publication
(10) Pub. No.: US 2004/0185537 A1
(43) Pub. Date: Sep. 23, 2004

(54) Novel fructosyltransferases

(75) Inventors: Sacha Adrianus Fokke Taco Van Hijum, Groningen (NL); Gerritdina Hendrika Van Geel-Schutten, Driebergen-Rijsenburg (NL); Lubbert Dijkhuizen, Zuidlaren (NL); Hakim Rahaoui, Amersfoort (NL)

Correspondence Address: YOUNG & THOMPSON, 745 SOUTH 23RD STREET 2ND FLOOR, ARLINGTON, VA, 22202

(73) Assignee: NEDERLANDSE ORGANISATIE VOOR TOEGEPAST NATUURWETENSCHAPPELIJK ONDERZOEK TNO, DELFT (NL)

(21) Appl. No.: 10/791,799

(22) Filed: Mar. 4, 2004

Related U.S. Application Data
(63) Continuation-in-part of application No. 09/995,587, filed on Nov. 29, 2001, now Pat. No. 6,730,502.
Application No. 09/995,587 is a continuation-in-part of application No. 09/604,958, filed on Jun. 28, 2000, now Pat. No. 6,635,460.

(30) Foreign Application Priority Data
May 25, 2000 (EP) 00201872.9

(51) Int. Cl. C12P 19/04; C12N 9/10; C12N 1/21

(52) U.S. Cl. 435/101; 536/123; 435/252.3; 435/193

(57) ABSTRACT

The present invention describes two novel proteins having fructosyltransferase activity. Both enzymes are derived from lactobacilli, which are food-grade micro-organisms with the Generally Recognized As Safe (GRAS) status. One of these proteins produces an inulin and fructo-oligosaccharides, while the other produces a levan and fructo-oligosaccharides. According to the invention lactobacilli capable of producing an inulin and/or a levan and/or fructo-oligosaccharides using one or both of the fructosyltransferases can be used as a probiotic or a symbiotic.

### CROSS-REFERENCE TO RELATED APPLICATIONS

This application is a continuation-in-part application of U.S. application Ser. No. 09/995,587, filed on Nov. 29, 2001, which is a continuation-in-part application of U.S. application Ser. No. 09/604,958, filed on Jun. 28, 2000, which claims priority from European Application No. 00201872.9 filed on May 25, 2000.

The present invention is in the field of enzymatic production of biomolecules. The invention is particularly concerned with two novel fructosyltransferases derived from lactobacilli and with a process for recombinant production of the enzymes and for the production of useful levans, inulins and fructo-oligosaccharides from sucrose.

### BACKGROUND OF THE INVENTION

Lactic acid bacteria (LAB) play an important role in the fermentative production of food and feed. Traditionally, these bacteria have been used for the production of for instance wine, beer, bread, cheese and yoghurt, and for the preservation of food and feed, e.g. olives, pickles, sausages, sauerkraut and silage. Because of these traditional applications, lactic acid bacteria are food-grade micro-organisms that posses the Generally Recognised As Safe (GRAS) status. Due to the different products which are formed during fermentation with lactic acid bacteria, these bacteria contribute positively to the taste, smell and preservation of the final product. The group of lactic acid bacteria encloses several genera such as Lactobacillus, Leuconostoc, Pediococcus, Streptococcus, etc.

In recent years also the health promoting properties of lactic acid bacteria have received much attention. They produce an abundant variety of exopolysaccharides (EPS's). These polysaccharides are thought to contribute to human health by acting as prebiotic substrates, nutraceuticals, cholesterol lowering agents or immunomodulants.

To date high molecular weight polysaccharides produced by plants (such as cellulose, starch and pectin), seaweeds (such as alginate and carrageenan) and bacteria (such as alginate, gellan and xanthan) are used in several industrial applications as viscosifying, stabilizing, emulsifying, gelling or water binding agents. Although all these polysaccharides are used as food additives, they originate from organisms not having the GRAS status. Thus they are less desirable than the exopolysaccharides of microorganisms, such as lactic acid bacteria, which have the GRAS status.

The exopolysaccharides produced by LAB can be divided in two groups, heteropolysaccharides and homopolysaccharides; these are synthesized by totally different mechanisms. The former consist of repeating units in which residues of different types of sugars are present and the latter consist of one type of monosaccharide. The synthesis of heteropolysaccharides by lactic acid bacteria, including lactobacilli, has been studied extensively in recent years. Considerably less information is available on the synthesis of homopolysaccharides from lactobacilli, although some studies have been performed. Homopolysaccharides with fructose as the constituent sugar can be divided into two groups, inulins and levans. Inulins consist of 2,1-linked β-fructofuranoside residues, whereas levans consist of 2,6-linked β-fructofuranoside residues. Both can be linear or branched. The size of bacterial levans can vary from 20 kDa up to several MDa. There is limited information on the synthesis of levans. In most detail this synthesis has been studied in Zymomonas mobilis and in Bacillus species. Within lactic acid bacteria, fructosyltransferases have only been studied in streptococci. So far no fructosyltransferases have been reported in lactobacilli.

In a recent report the Lactobacillus reuteri strain LB 121 was found to produce both a glucan and a fructan when grown on sucrose, but only a fructan when grown on raffinose (van Geel-Schutten, G. H. et al., Appl. Microbiol. Biotechnol. (1998) 50, 697-703). In another report the glucan and fructan were characterised by their molecular weights (of 3,500 and 150 kDa respectively) and the glucan was reported to be highly branched with a unique structure consisting of a terminal, 4-substituted, 6-substituted, and 4,6-disubstituted α-glucose in a molar ratio 1.1:2.7:1.5:1.0 (van Geel-Schutten, G. H. et al., Appl. Environ. Microbiol. (1999) 65, 3008-3014). The fructan was identified as a linear (2→6)-β-D-fructofuranan (also called a levan). This was the first example of fructan synthesis by a Lactobacillus species.

### SUMMARY OF THE INVENTION

Two novel genes encoding enzymes having fructosyltransferase activity have now been found in Lactobacillus reuteri, and their amino acid sequences have been determined. These are the first two enzymes identified in a Lactobacillus species capable of producing a fructan. One of the enzymes is an inulosucrase which produces a high molecular weight (>10⁷ Da) fructan containing β(2-1) linked fructosyl units and fructo-oligosaccharides, while the other is a levansucrase which produces a fructan containing β(2-6) linked fructosyl units. The invention thus pertains to the enzymes, to DNA encoding them, to recombinant cells containing such DNA and to their use in producing carbohydrates, as defined in the appending claims.

### DESCRIPTION OF THE INVENTION

It was found according to the invention that one of the novel fructosyltransferases (FTFA; an inulosucrase) produces a high molecular weight inulin with β(2-1) linked fructosyl units and fructo-oligosaccharides. The fructo-oligosaccharides synthesis was also observed in certain Lactobacillus strains, in particular in certain strains of Lactobacillus reuteri. However, the inulin has not been found in Lactobacillus reuteri culture supernatants, but only in extracts of E. coli cells expressing the above-mentioned fructosyltransferase. This inulosucrase consists of either 798 amino acids (2394 nucleotides) or 789 amino acids (2367 nucleotides) depending on the potential start codon used. The molecular weight (MW) deduced of the amino acid sequence of the latter form is 86 kDa and its isoelectric point is 4.51, at pH 7.

The amino acid sequence of the inulosucrase is shown in SEQ ID No. 1 (FIG. 1, amino acid residues 1-789). As mentioned above, the nucleotide sequence contains two putative start codons leading to either a 2394 (see SEQ ID No. 3) or 2367 (see SEQ ID No. 2) nucleotide form of the inulosucrase. Both putative start codons are preceded by a putative ribosome binding site, GGGG (located 12 base pairs upstream its start codon) or AGGA (located 14 base pairs upstream its start codon), respectively (see FIG. 1 and SEQ ID No. 4).

The present invention covers a protein having inulosucrase activity with an amino acid identity of at least 65%, preferably at least 75%, and more preferably at least 85%, compared to the amino acid sequence of SEQ ID No. 1. The invention also covers a part of a protein with at least 15 contiguous amino acids which are identical to the corresponding part of the amino acid sequence of SEQ ID No. 1.

Fructosyltransferases have been found in several bacteria such as Zymomonas mobilis, Erwinia amylovora, Acetobacter amylovora, Bacillus polymyxa, Bacillus amyloliquefaciens, Bacillus stearothermophilus, and Bacillus subtilis. In lactic acid bacteria this type of enzyme previously has only been found in some streptococci. Most bacterial fructosyltransferases have a molecular mass of 50-100 kDa (with the exception of the fructosyltransferase found in Streptococcus salivarius which has a molecular mass of 140 kDa). Amino acid sequence alignment revealed that the novel inulosucrase of lactobacilli has high homology with fructosyltransferases originating from Gram positive bacteria, in particular with Streptococcus enzymes. The highest homology (FIG. 2) was found with the SacB enzyme of Streptococcus mutans Ingbritt A (62% identity within 539 amino acids).

Certain putative functions based on the alignment and site-directed mutagenesis studies can be ascribed to several amino acids of the novel inulosucrase. Asp-263, Glu-330, Asp-415, Glu-431, Asp-511, Glu-514, Arg-532 and/or Asp-551 of the amino acid sequence of SEQ ID No. 1 are identified as putative catalytic residues. Noteworthy, a hydrophobicity plot according to Kyte and Doolittle (1982) J. Mol. Biol. 157, 105-132 suggests that the novel inulosucrase contains a putative signal sequence according to the Von Heijne rule. The putative signal peptidase site is located between Gly at position 21 and Ala at position 22. Furthermore, it is striking that the C-terminal amino acid sequence of the novel inulosucrase contains a putative cell wall anchor amino acid signal LPXTG (SEQ ID No. 5) and a 20-fold repeat of the motif PXX (residues 690-749 of SEQ ID NO: 1) (see FIG. 1), where P is proline and X is any other amino acid. In 15 out of 20 repeats, however, the motif is PXT. This motif has so far not been reported in proteins of prokaryotic and eukaryotic origin.

A nucleotide sequence encoding any of the above mentioned proteins, mutants, variants or parts thereof is also a subject of the invention. Furthermore, the nucleic acid sequences corresponding to expression-regulating regions (promoters, enhancers, terminators) of at least 30 contiguous nucleic acids contained in the nucleic acid sequence (-67)-(-1) or 2367-2525 of SEQ ID No. 4 (see also FIG. 1) can be used for homologous or heterologous expression of genes. Such expression-regulating sequences are operationally linked to a polypeptide-encoding nucleic acid sequence such as the genes of the fructosyltransferase according to the invention. A nucleic acid construct comprising the nucleotide sequence operationally linked to an expression-regulating nucleic acid sequence is also covered by the invention.

A recombinant host cell, such as a mammalian (with the exception of human), plant, animal, fungal or bacterial cell, containing one or more copies of the nucleic acid construct mentioned above is an additional subject of the invention. The inulosucrase gene (starting at nucleotide 41) has been cloned in an E. coli expression vector under the control of an ara promoter in E. coli Top10. E. coli Top10 cells expressing the recombinant inulosucrase hydrolyzed sucrose and synthesized fructan material. SDS-PAGE of arabinose induced E. coli Top10 cell extracts suggested that the recombinant inulosucrase has a molecular weight of 80-100 kDa, which is in the range of other known fructosyltransferases and in line with the molecular weight of 86 kDa deduced of the amino acid sequence depicted in FIG. 1.

The invention further covers an inulosucrase according to the invention which, in the presence of sucrose, produces a inulin having β(2-1)-linked D-fructosyl units and fructo-oligosaccharides. Two different types of fructans, inulins and levans, exist in nature. Surprisingly, the novel inulosucrase expressed in E. coli Top10 cell synthesizes a high molecular weight (>10⁷ Da) inulin and fructo-oligosaccharides, while in Lactobacillus reuteri culture supernatants, in addition to the fructo-oligosaccharides, a levan and not an inulin is found. This discrepancy can have several explanations: the inulosucrase gene may be silent in Lactobacillus reuteri, or may not be expressed in Lactobacillus reuteri under the conditions tested, or the inulosucrase may only synthesize fructo-oligosaccharides in its natural host, or the inulin polymer may be degraded shortly after synthesis, or may not be secreted and remains cell-associated, or the inulosucrase may have different activities in Lactobacillus reuteri and E. coli Top 10 cells.

It was furthermore found according to the invention that certain lactobacilli, in particular Lactobacillus reuteri, possess another fructosyltransferase, a levansucrase (FTFB), in addition to the inulosucrase described above. The N-terminal amino acid sequence of the fructosyltransferase purified from Lactobacillus reuteri supernatant was found to be QVESNNYNGVAEVNTERQANGQI (residues 2-24 of SEQ ID No. 6). Furthermore, three internal sequences were identified, namely (M)(A)HLDVWDSWPVQDP(V) (SEQ ID No. 7), NAGSIFGT(K) (SEQ ID No. 8), V(E)(E)VYSPKVSTLMASDEVE (SEQ ID No. 9). The N-terminal amino acid sequence could not be identified in the deduced inulosucrase sequence. Also the amino acid sequences of the three internal peptide fragments of the purified fructosyltransferase were not present in the putative inulosucrase sequence. Evidently, the inulosucrase gene does not encode the purified fructosyltransferase synthesizing the levan. The complete amino acid sequence of the levansucrase is shown in SEQ ID No. 11 and the nucleotide sequence is shown in SEQ ID No. 10. The levansucrase comprises a putative membrane anchor (see amino acids 761-765 in SEQ ID No. 11) and a putative membrane spanning domain (see amino acids 766-787 in SEQ ID No. 11). The fructan produced by the levansucrase was identified in the Lactobacillus reuteri culture supernatant as a linear (2→6)-β-D-fructofuranan with a molecular weight of 150 kDa. The purified enzyme also produces this fructan.

Additionally, the invention thus covers a protein having levansucrase activity with an amino acid identity of at least 65%, preferably at least 75%, and more preferably at least 85%, compared to the amino acid sequence of SEQ ID NO. 11. The second novel fructosyltransferase produces a high molecular weight fructan with β(2-6) linked fructosyl units with sucrose or raffinose as substrate. The invention also covers a part of a protein with least 15 contiguous amino acids, which are identical to the corresponding part of the amino acid sequence of SEQ ID No. 11. A nucleotide sequence encoding any of the above-mentioned proteins, mutants, variants or parts thereof is a subject of the invention as well as a nucleic acid construct comprising the nucleotide sequence mentioned above operationally linked to an expression-regulating nucleic acid sequence. A recombinant host cell, such as a mammalian (with the exception of human), plant, animal, fungal or bacterial cell, containing one or more copies of the nucleic acid construct mentioned above is an additional subject of the invention. The invention further covers a protein according to the invention which, in the presence of sucrose, produces a fructan having β(2-6)-linked D-fructosyl units.

In addition to the percentage of amino acid identity as referred to above, the proteins according to the invention preferably have a minimum degree of homology of at least 85%, preferably at least 90%, more preferably at least 95, 96, 97, even at least 98 or 99% with a given sequence, for example with amino acid sequences 22-789 of SEQ ID No. 1 or 22-792 of SEQ ID No. 11. A degree of homology as used herein and as commonly used in the art is equivalent to a degree of similarity as defined below, which means that two amino acids which are similar in both size and electronic structure but not identical, are treated as “conserved” substitutions of each other and thus are considered to fulfil the homology requirement. Conserved substitutions include substitutions among valine-leucine-isoleucine-methionine, alanine-valine, alanine-glycine, serine-threonine, phenylalanine-tyrosine-tryptophan, lysine-arginine-histidine, asparagine-glutamine-histidine, aspartic acid-glutamic acid. Methods to determine identity and similarity are codified in publicly available computer programs. Preferred computer program methods to determine identity and similarity between two sequences include e.g. the GCG program package (Devereux, J., et al., Nucleic Acids Research 12 (1):387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990).

The invention also pertains to a process of producing an inulin-type and/or a levan-type of fructan as described above using fructosyltransferases according to the invention and a suitable fructose source such as sucrose, stachyose, raffinose or a fructo-oligosaccharide. The fructans may either be produced by Lactobacillus strains or recombinant host cells according to the invention containing one or both fructosyl transferases or by a fructosyltransferase enzyme isolated by conventional means from the culture of fructosyltransferase-positive lactobacilli, especially a Lactobacillus reuteri, or from a recombinant organism containing the fructosyltransferase gene or genes.

Where the fructans are produced by Lactobacillus strains, the strain may advantageously be in a non-growth state. This allows the production of the fructans to proceed without the necessity of supplying nutrients and other materials for supporting growth of the strains. In particular, the production can be performed by merely subjecting the sucrose or other fructose source such as raffinose to a medium containing the strains and withdrawing polysaccharides from the medium, or using the medium together with the polysaccharides to produce the desired end product. In a particular embodiment, the Lactobacillus strain may be immobilized on a carrier such as solid particles, filters, reactor walls and the like. The strains to be used may be native or recombinant, as long as they are capable of expressing at least one of the inulosucrase and levansucrase enzymes as described above. In addition, they may also contain an active glucansucrase enzyme and at the same time produce glucans. The polysaccharide production can be performed at neutral to slightly acidic conditions, in particular at a pH between 4 and 7, at temperatures between 25 and 60° C., preferably between 30 and 55° C. The fructose donor such as sucrose is preferably present at a level between 10 and 50 g/l.

Additionally, the invention concerns a process of producing fructo-oligosaccharides containing the characteristic structure of the fructans described above using a Lactobacillus strain or a recombinant host cell according to the invention containing one or both fructosyltransferases or an isolated fructosyltransferase according to the invention. There is a growing interest in oligosaccharides derived from homopolysaccharides, for instance for prebiotic purposes. Several fructo- and gluco-oligosaccharides are known to stimulate the growth of bifidobacteria in the human colon. Fructo-oligosaccharides produced by the fructosyltransferase described above are also part of the invention. Another way of producing fructo-oligosaccharides is by hydrolysis of the fructans described above. This hydrolysis can be performed by known hydrolysis methods such as enzymatic hydrolysis with enzymes such as levanase or inulinase or by acid hydrolysis. The fructo-oligosaccharides can also be produced in the presence of a fructosyltransferase according to the invention and an acceptor molecule such as lactose or maltose. The fructo-oligosaccharides to be produced according to the invention preferably contain at least 2, more preferably at least 3, up to about 20 anhydrofructose units, optionally in addition to one or more other (glucose, galactose, etc.) units. These fructo-oligosaccharides are useful as prebiotics, and can be administered to a mammal in need of improving the bacterial status of the colon.

The invention also concerns chemically modified fructans and fructo-oligosaccharides based on the fructans described above. Chemical modification can be achieved by oxidation, such as hypochlorite oxidation resulting in ring-opened 2,3-dicarboxy-anhydrofructose units (see e.g. EP-A-427349), periodate oxidation resulting in ring-opened 3,4-dialdehyde-anhydrofructose units (see e.g. WO 95/12619), which can be further oxidized to (partly) carboxylated units (see e.g. WO 00/26257), nitroxyl (TEMPO)-mediated oxidation resulting in 1-carboxy-anhydrofructose units (in levans) or 6-carboxy-anhydrofructose units (in inulins) (see e.g. WO 95/07303). The oxidized fructans have improved water-solubility, altered viscosity and a retarded fermentability and can be used as metal-complexing agents, detergent additives, strengthening additives, bioactive carbohydrates, emulsifiers and water binding agents. They can also be used as starting materials for further derivatisation such as cross-linking and the introduction of hydrophobes. Oxidized fructans coupled to amino compounds such as proteins, or fatty acids can be used as emulsifiers and stabilizers. (Partial) hydrolysis of fructans according to the invention and modified fructans according to the invention results in fructo-oligosaccharides, which can be used as bioactive carbohydrates or prebiotics. The oxidized fructans of the invention preferably contain 0.05-1.0 carboxyl groups per anhydrofructose unit, e.g. as 6- or 1-carboxyl units. For use as e.g. a water treatment chemical serving as an alternative for acrylate-based products (e.g. flocculant or anti-scaling) or paper additive (aldehyde-containing polymer) the preferred degree of oxidation (aldehyde or carboxyl content) is between 0.1 and 0.8 aldehyde/carboxyl groups per anhydrofructose unit and the preferred degree of polymerization is between 37 and 12,500 anhydrofructose units (molecular weight between about 6 and 2,000 kDa. Alternatively, negative charges can be introduced into the molecule by, e.g., carboxymethylation.

Another type of chemical modification is phosphorylation, as described in O. B. Wurzburg (1986) Modified Starches: properties and uses. CRC Press Inc., Boca Raton, 97-112. One way to achieve this modification is by dry heating fructans with a mixture of monosodium and disodium hydrogen phosphate or with tripolyphosphate. The phosphorylated fructans are suitable as wet-end additives in papermaking, as binders in paper coating compositions, as warp sizing-agents, and as core binders for sand molds for metal casting. A further type of derivatisation of the fructans is acylation, especially acetylation using acetic or propionic anhydride, resulting in products suitable as bleaching assistants and for the use in foils. Acylation with e.g. alkenyl succinic anhydrides or (activated) fatty acids results in surface-active products suitable as e.g. surfactants, emulsifiers, and stabilizers.

Hydroxyalkylation, carboxymethylation, epoxyalkylation and aminoalkylation are other methods of chemical derivatisation of the fructans. Hydroxyalkylation is commonly performed by base-catalysed reaction with alkylene oxides, such as ethylene oxide, propylene oxide or epichlorohydrine; the hydroxyalkylated products have improved solubility and viscosity characteristics. Carboxymethylation is achieved by reaction of the fructans with monochloroacetic acid or its alkali metal salts and results in anionic polymers suitable for various purposes including crystallisation inhibitors, and metal complexants. Epoxyalkylation can be achieved using diepoxy compounds such as diglycidyl ether and bis-epoxyalkanes, but is preferably performed by first reacting the fructan with an epoxyalkene such as 1,2-epoxy-5-hexene or allyl glycidyl ether followed by oxidation of the ethylenic bond to an epoxide using a peracid or hydrogen peroxide as described in WO 01/87986. The epoxyalkylated fructans can be used for coupling compounds having e.g. carboxyl, amino or mercapto groups, including peptides and proteins, for producing crosslinked fructans. Aminoalkylation can be achieved by reaction of the fructans with alkylene imines, haloalkyl amines or amino-alkylene oxides, or by reaction of epichlorohydrin adducts of the fructans with suitable amines. These products can be used as cationic polymers in a variety of applications, especially as a wet-end additive in paper making to increase strength, for filler and fines retention, and to improve the drainage rate of paper pulp. Other potential applications include textile sizing and waste water purification.

The above mentioned modifications can be used either separately or in combination depending on the desired product. Furthermore, the degree of chemical modification is variable and depends on the intended use. If necessary 100% modification, i.e. modification of all anhydrofructose units can be performed. However, partial modification, e.g. from 1 modified anhydrofructose unit per 100 up to higher levels, will often be sufficient in order to obtain the desired effect. The modified fructans have a DP (degree of polymerization) of at least 100, preferably at least 1000 units. Use of partially modified fructans containing, e.g., (negative) charges as well as aldehyde moieties are particularly useful in paper applications and textile treatment. These have a degree of substitution for aldehyde groups between 1 and 50%, preferably between 5 and 40%, and a degree of substitution for carboxyl or other charged groups between 1 and 50%, preferably between 5 and 40%.

Use of a Lactobacillus strain capable of producing a levan, inulin or fructo-oligosaccharides or a mixture thereof, as a probiotic, is also covered by the invention. Preferably, the Lactobacillus strain is also capable of producing a glucan, especially an 1,4/1,6-α-glucan as referred to above. The efficacy of some Lactobacillus reuteri strains as a probiotics has been demonstrated in various animals such as for instance poultry and humans. The administration of some Lactobacillus reuteri strains to pigs resulted in significantly lower serum total and LDL-cholesterol levels, while in children Lactobacillus reuteri is used as a therapeutic agent against acute diarrhea. For this and other reasons Lactobacillus reuteri strains, which were not reported to produce the glucans or fructans described herein, have been supplemented to commercially available probiotic products. The mode of action of Lactobacillus reuteri as a probiotic is still unclear. Preliminary studies indicated that gut colonization by Lactobacillus reuteri may be of importance. According to the invention, it was found that the mode of action of Lactobacillus reuteri as a probiotic may reside partly in the ability to produce polysaccharides. Lactobacillus strains, preferably Lactobacillus reuteri strains, and more preferably Lactobacillus reuteri strain LB 121 and other strains containing one or more fructosyltransferase genes encoding proteins capable of producing inulins, levans and/or fructo-oligosaccharides can thus advantageously be used as a probiotic. They can also, together with these polysaccharides, be used as a symbiotic (instead of the term symbiotic, the term synbiotic can also be used). In that respect another part of the invention concerns a probiotic or symbiotic composition containing a Lactobacillus strain capable of producing an inulin, a levan or fructo-oligosaccharides and/or a glucan or a mixture thereof, said production being performed according to the process according to the invention. The probiotic or symbiotic compositions of the invention may be directly ingested with or without a suitable vehicle or used as an additive in conjunction with foods. They can be incorporated into a variety of foods and beverages including, but not limited to, yogurts, ice creams, cheeses, baked products such as bread, biscuits and cakes, dairy and dairy substitute foods, confectionery products, edible oil compositions, spreads, breakfast cereals, juices and the like.

Furthermore, the invention pertains to a process of improving the microbial status in the mammalian colon comprising administering an effective amount of a Lactobacillus strain capable of producing an oligosaccharide or polysaccharide according ot the invention and to a process of improving the microbial status of the mammalian colon comprising administering an effective amount of an oligosaccharide or polysaccharide produced according to the process according to the invention.

### EXAMPLES

### Example 1

Isolation of DNA from Lactobacillus reuteri, nucleotide sequence analysis of the inulosucrase (ftfA) gene, construction of plasmids for expression of the inulosucrase gene in E. coli Top10, expression of the inulosucrase gene in E. coli Top10 and identification of the produced polysaccharides produced by the recombinant enzyme. General procedures for cloning, DNA manipulations and agarose gel electrophoresis were essentially as described by Sambrook et al. (1989) Molecular cloning: a laboratory manual, 2nd ed. Cold Spring Harbour Laboratory Press, Cold Spring Harbour, N.Y. Restriction endonuclease digestions and ligations with T4 DNA ligase were performed as recommended by the suppliers. DNA was amplified by PCR techniques using ampliTAQ DNA polymerase (Perkin Elmer) or Pwo DNA polymerase. DNA fragments were isolated from agarose gels using the Qiagen extraction kit (Qiagen GMBH), following the instructions of the suppliers. Lactobacillus reuteri strain 121 (LMG 18388) was grown at 37° C. in MRS medium (DIFCO) or in MRS-s medium (MRS medium containing 100 g/l sucrose instead of 20 g/l glucose). When fructo-oligosaccharides production was investigated phosphate was omitted and ammonium citrate was replaced by ammonium nitrate in the MRS-s medium. E. coli strains were grown aerobically at 37° C. in LB medium, where appropriate supplemented with 50 μg/ml ampicillin (for selection of recombinant plasmids) or with 0.02% (w/v) arabinose (for induction of the inulosucrase gene).

Total DNA of Lactobacillus reuteri was isolated according to Verhasselt et al. (1989) FEMS Microbiol. Lett. 59, 135-140 as modified by Nagy et al. (1995) J. Bacteriol. 177, 676-687.

The inulosucrase gene was identified by amplification of chromosomal DNA of Lactobacillus reuteri with PCR using degenerated primers (5ftf, 6ftfi, and 12ftfi, see table 1) based on conserved amino acid sequences deduced from different bacterial fructosyltranferase genes (SacB of Bacillus amyloliquefaciens, SacB of Bacillus subtilis, Streptococcus mutans fructosyltransferase and Streptococcus salivarius fructosyltransferase, see FIG. 4) and Lactobacillus reuteri DNA as template. Using primers 5ftf and 6ftfi, an amplification product with the predicted size of about 234 bp was obtained (FIG. 5A). This 234 bp fragment was cloned in E. coli JM109 using the pCR2.1 vector and sequenced. Transformations were performed by electroporation using the BioRad gene pulser apparatus at 2.5 kV, 25 μF and 200 Ω, following the instructions of the manufacturer. Sequencing was performed according to the method of Sanger et al. (1977) Proc. Natl. Acad. Sci. USA 74, 5463-5467. Analysis of the obtained sequence data confirmed that part of a fructosyltransferase (ftf) gene had been isolated. The 234 bp amplified fragment was used to design primers 7ftf and 8ftfi (see table 1). PCR with the primers 7ftf and 12ftfi gave a product of the predicted size of 948 bp (see FIG. 5B); its sequence showed clear similarity with previously characterized fructosyltransferase genes. The 948 bp amplified fragment was used to design the primers ftfAC1(i) and ftfAC2(i) (see table 1) for inverse PCR. Using inverse PCR techniques a 1438 bp fragment of the inulosucrase gene was generated, including the 3′ end of the inulosucrase gene (see FIG. 5C). The remaining 5′ fragment of the inulosucrase gene was isolated with a combination of standard and inverse PCR techniques. Briefly, Lactobacillus reuteri DNA was cut with restriction enzyme XhoI and ligated. PCR with the primers 7ftf and 8ftfi, using the ligation product as a template, yielded a 290 bp PCR product which was cloned into pCR2.1 and sequenced. This revealed that primer 8ftfi had annealed aspecifically as well as specifically yielding the 290 bp product (see FIG. 5D).

At this time, the N-terminal amino acid sequence of a fructosyltransferase enzyme (FTFB) purified from the Lactobacillus reuteri strain 121 was obtained. This sequence consisted of the following 23 amino acids: QVESNNYNGVAEVNTERQANGQI (residues 2-24 of SEQ ID No. 6). The degenerated primer 19ftf (YNGVAEV) (residues 8-14 of SEQ ID No. 6) was designed on the basis of a part of this N-terminal peptide sequence and primer 20ftfi was designed on the 290 bp PCR product. PCR with primers 19ftf and 20ftfi gave a 754 bp PCR product (see FIG. 5E), which was cloned into pCR2.1 and sequenced. Both DNA strands of the entire fructosyltransferase gene were double sequenced. In this way the sequence of a 2.6 kb region of the Lactobacillus reuteri DNA, containing the inulosucrase gene and its surroundings were obtained.

The plasmids for expression of the inulosucrase gene in E. coli Top10 were constructed as described hereafter. A 2414 bp fragment, containing the inulosucrase gene starting at the first putative start codon at position 41, was generated by PCR, using primers ftfA1 and ftfA2i. Both primers contained suitable restriction enzyme recognition sites (a NcoI site at the 5′end of ftfA1 and a BglII site at the 3′end of ftfA2i). PCR with Lactobacillus reuteri DNA, Pwo DNA polymerase and primers ftfA1 and ftfA2i yielded the complete inulosucrase gene flanked by NcoI and BglII restriction sites. The PCR product with blunt ends was ligated directly into pCRbluntII-Topo. Using the NcoI and BglII restriction sites, the putative ftfA gene was cloned into the expression vector pBAD, downstream of the inducible arabinose promoter and in frame upstream of the Myc epitope and the His tag. The pBAD vector containing the inulosucrase gene (pSVH101) was transformed to E. coli Top10 and used to study inulosucrase expression. Correct construction of plasmid containing the complete inulosucrase gene was confirmed by restriction enzyme digestion analysis and by sequence analysis, showing an in frame cloning of the inulosucrase gene using the ribosomal binding site provided by the pBAD vector and the first putative start codon (at position 41) of inulosucrase (see FIG. 1).

Plasmid DNA of E. coli was isolated using the alkaline lysis method of Birnboim and Doly (1979) Nucleic Acids Res. 7, 1513-1523 or with a Qiagen plasmid kit following the instructions of the supplier. Cells of E. coli Top10 with pSVH101 were grown overnight in LB medium containing 0.02% (w/v) arabinose and were harvested by centrifugation. The pellet was washed with 25 mM sodium acetate buffer pH 5.4 and the suspension was centrifuged again. Pelleted cells were resuspended in 25 mM sodium acetate buffer pH 5.4. Cells were broken by sonication. Cell debris and intact cells were removed by centrifugation for 30 min at 4° C. at 10,000×g and the resulting cell free extract was used in the enzyme assays.

The fructosyltranferase activities were determined at 37° C. in reaction buffer (25 mM sodium acetate, pH 5.4, 1 mM CaCl₂, 100 g/l sucrose) by monitoring the release of glucose from sucrose, by detecting fructo-oligosaccharides or by determining the amount of fructan polymer produced using E. coli cell free extracts or Lactobacillus reuteri culture supernatant as enzyme source. Sucrose, glucose and fructose were determined enzymatically using commercially available kits.

Fructan production by Lactobacillus reuteri was studied with cells grown in MRS-s medium. Product formation was also studied with cell-free extracts of E. coli containing the novel inulosucrase incubated in reaction buffer (1 mg protein/10 ml buffer, incubated overnight at 37° C.). Fructans were collected by precipitation with ethanol. ¹H-NMR spectroscopy and methylation analysis were performed as described by van Geel-Schutten et al. (1999) Appl. Environ. Microbiol. 65, 3008-3014. The molecular weights of the fructans were determined by high performance size exclusion chromatography coupled on-line with a multi angle laser light scattering and a differential refractive index detector. Fructo-oligosaccharide synthesis was studied in Lactobacillus reuteri culture supernatants and in extracts of E. coli cells containing the novel inulosucrase incubated in reaction buffer (1 mg protein/10 ml buffer, incubated overnight at 37° C.). Glucose and fructose were determined enzymatically as described above and fructo-oligosaccharides produced were analyzed using a Dionex column. The incubation mixtures were centrifuged for 30 min at 10,000×g and diluted 1:5 in a 100% DMSO solution prior to injection on a Dionex column. A digest of inulin (DP1-20) was used as a standard. Separation of compounds was achieved with anion-exchange chromatography on a CarboPac Pa1 column (Dionex) coupled to a CarboPac PA1 guard column (Dionex). Using a Dionex GP50 pump the following gradient was generated: % eluent B is 5% (0 min); 35% (10 min); 45% (20 min); 65% (50 min); 100% (54-60 min); 5% (61-65 min). Eluent A was 0.1 M NaOH and eluent B was 0.6 M NaAc in a 0.1 M NaOH solution. Compounds were detected using a Dionex ED40 electrochemical detector with an AU working electrode and a Ag/AgCl reference-electrode with a sensitivity of 300 nC. The pulse program used was: +0.1 Volt (0-0.4 s); +0.7 Volt (0.41-0.60 s); −0.1 Volt (0.61-1.00 s). Data were integrated using a Perkin Elmer Turbochrom data integration system. A different separation of compounds was done on a cation exchange column in the calcium form (Benson BCX4). As mobile phase C a-EDTA in water (100 ppm) was used. The elution speed was 0.4 ml/min at a column temperature of 85° C. Detection of compounds was done by a refractive index (Jasco 830-RI) at 40° C. Quantification of compounds was achieved by using the software program Turbochrom (Perkin Elmer).

SDS-PAGE was performed according to Laemmli (1970) Nature 227, 680-685 using 7.5% polyacrylamide gels. After electrophoresis gels were stained with Coomassie Brilliant Blue or an activity staining (Periodic Acid Schiff, PAS) was carried out as described by Van Geel-Schutten et al. (1999) Appl. Environ. Microbiol. 65, 3008-3014.

TABLE 1

Nucleotide sequence of primers used in PCR reactions
to identify the inulosucrase gene.

| | | |
|---|---|---|
| Primer | Location | Nucleotide sequence |

| | | |
|---|---|---|
| name | (bp) | (and SEQ ID No) |

| | | |
|---|---|---|
| ftfAC1 | 1176 | CTG-ATA-ATA-ATG-GAA-ATG-TAT-CAC |
| | | (SEQ ID No. 12) |
| ftfAC2i | 1243 | CAT-GAT-CAT-AAG-TTT-GGT-AGT-AAT-AG |
| | | (SEQ ID No. 13) |
| ftfac1 | 1176 | GTG-ATA-CAT-TTC-CAT-TAT-TAT-CAG |
| | | (SEQ ID No. 14) |
| ftfAC2 | 1243 | CTA-TTA-CTA-CCA-AAC-TTA-TGA-TCA-TG |
| | | (SEQ ID No. 15) |
| ftfA1 | | CCA-TGG-CCA-TGG-TAG-AAC-GCA-AGG-AAC |
| | | ATA-AAA-AAA-TG |
| | | (SEQ ID No. 16) |
| ftfA2i | | AGA-TCT-AGA-TCT-GTT-AAA-TCG-ACG-TTT- |
| | | GTT-AAT-TTC-TG |
| | | (SEQ ID No. 17) |
| 5ftf | 845 | GAY-GTN-TGG-GAY-WSN-TGG-GCC |
| | | (SEQ ID No. 18) |
| 6ftfi | 1052 | GTN-GCN-SWN-CCN-SWC-CAY-TSY-TG |
| | | (SEQ ID No. 19) |
| 7ftf | 1009 | GAA-TGT-AGG-TCC-AAT-TTT-TGG-C |
| | | (SEQ ID No. 20) |
| 8ftfi | 864 | CCT-GTC-CGA-ACA-TCT-TGA-ACT-G |
| | | (SEQ ID No. 21) |
| 12ftfi | 1934 | ARR-AAN-SWN-GGN-GCV-MAN-GTN-SW |
| | | (SEQ ID No. 22) |
| 19ftf | 1 | TAY-AAY-GGN-GTN-GCN-GAR-GTN-AA |
| | | (SEQ ID No. 23) |
| 20ftfi | 733 | CCG-ACC-ATC-TTG-TTT-GAT-TAA-C |
| | | (SEQ ID No. 24) |

Listed from left to right are: primer name (i, inverse primer), location (in bp) in ftfA and the sequence from 5′ to 3′ according to IUB group codes (N=any base;M=A or C; R=A or G; W=A or T; S=C or G; Y=C or T; K=G or T; B=not A; D=not C; H=not G; and V=not T).

### Example 2

Purification and amino acid sequencing of the levansucrase (FTFB).

Protein purification

Samples were taken between each step of the purification process to determine the enzyme activity (by glucose GOD-Perid method) and protein content (by Bradford analysis and acrylamide gel electrophoresis). Collected chromatography fractions were screened for glucose liberating activity (GOD-Perid method) to determine the enzyme activity.

One liter of an overnight culture of LB121 cells grown on MRS medium containing 50 grams per liter maltose was centrifuged for 15 min. at 10,000×g. The supernatant was precipitated with 1.5 liter of a saturated ammonium sulphate solution. The ammonium sulphate solution was added at a rate of 50 ml/min. under continuous stirring. The resulting 60% (w/v) ammonium sulphate solution was centrifuged for 15 min. at 10,000×g. The precipitate was resuspended in 10 ml of a sodium phosphate solution (10 mM, pH 6.0) and dialyzed overnight against 10 mM sodium phosphate, pH 6.0.

A hydroxylapatite column was washed with a 10 mM sodium phosphate solution pH 6.0; the dialyzed sample was loaded on the column. After eluting the column with 200 mM sodium phosphate, pH 6.0 the eluted fractions were screened for glucose releasing activity and fractions were pooled for phenyl superose (a hydrophobic interactions column) chromatography.

The pooled fractions were diluted 1:1 (v:v) with 25 mM sodium acetate, 2 M ammonium sulphate, pH 5.4 and loaded on a phenyl superose column (washed with 25 mM sodium acetate, 1 M ammonium sulphate, pH 5.4). In a gradient from 25 mM sodium acetate, 1 M ammonium sulphate, pH 5.4 (A) to 25 mM sodium acetate, pH 5.4 (B) fractions were collected from 35% B to 50% B.

Pooled fractions from the phenyl superose column were loaded on a gel filtration (superdex) column and eluted by a 25 mM acetate, 0.1 M sodium chloride, pH 5.4 buffer. The superdex fractions were loaded on a washed (with 25 mM sodium acetate, pH 5.4) Mono Q column and eluted with 25 mM sodium acetate, 1 M sodium chloride, pH 5.4. The fractions containing glucose liberating activity were pooled, dialyzed against 25 mM sodium acetate, pH 5.4, and stored at −20° C.

A levansucrase enzyme was purified from LB 121 cultures grown on media containing maltose using ammonium sulfate precipitation and several chromatography column steps (table 2). Maltose (glucose-glucose) was chosen because both glucansucrase and levansucrase can not use maltose as substrate. LB121 will grow on media containing maltose but will not produce polysaccharide. From earlier experiments it was clear that even with harsh methods the levansucrase enzyme could not be separated from its product levan. These harsh methods included boiling the levan in a SDS solution and treating the levan with HCl and TFA. No levanase enzyme was commercially available for the enzymatic breakdown of levan. Only a single levansucrase was detected in maltose culture supernatants. In order to prove that the enzyme purified from maltose culture supernatant is the same enzyme which is responsible for the levan production during growth on raffinose, biochemical and biophysical tests were performed.

TABLE 2
Purification of the Lactobacillus reuteri
LB 121 levansucrase (FTFB) enzyme.

| | | Total | Specific | | |
|---|---|---|---|---|---|
| | Protein | Activity | Activity | Purification | Yield |
| Step | (mg) | (U) | (U/mg) | (fold) | (%) |
| Supernatant | 128 | 64 | 0.5 | 1 | 100 |
| Ammonium sulfate | 35.2 | 42 | 1.2 | 2.4 | 65.6 |
| precipitation (65%) | | | | | |
| Hydroxyl apatite | 1.5 | 30.6 | 20.4 | 40.8 | 47.8 |
| Phenyl superose | 0.27 | 23 | 85 | 170 | 36 |
| Gel Filtration | 0.055 | 10 | 182 | 360 | 16 |
| MonoQ | 0.0255 | 4 | 176 | 352 | 6 |

Amino acid sequencing of FTFB

A 5% SDS-PAA gel was allowed to “age” overnight in order to reduce the amount of reacting chemical groups in the gel. Reaction of chemicals in the PAA gel (TEMED and ammonium persulphate) with proteins can cause some undesired effects, such as N-terminal blocking of the protein, making it more difficult to determine the protein amino acid composition. 0.1 mM thioglycolic acid (scavenger to reduce the amount of reactive groups in the PAA gel material) was added to the running buffer during electrophoresis.

In order to determine the amino acid sequence of internal peptides of protein bands running in a SDS-PAA gel, protein containing bands were cut out of the PAA gel. After fractionating the protein by digestion with chymotrypsin the N-terminal amino acid sequences of the digested proteins were determined (below).

N-terminal sequencing was performed by Western blotting of the proteins from the PAA gel to an Immobilon PVDF membrane (Millipore/ Waters Inc.) at 0.8 mA/cm² for 1 h. After staining the PVDF membrane with Coomassie Brilliant Blue without adding acetic acid (to reduce N-terminal blocking) and destaining with 50% methanol, the corresponding bands were cut out of the PVDF membrane for N-terminal amino acid sequence determination.

Amino acid sequence determination was performed by automated Edman degradation as described by Koningsberg and Steinman (1977) The proteins (third edition) volume 3, 1-178 (Neurath and Hill, eds.). The automated equipment for Edman degradation was an Applied Biosystems model 477A pulse-liquid sequenator described by Hewick et al. (1981), J. Biol. Chem. 15, 7990-7997 connected to a RP-HPLC unit (model 120A, Applied Biosystems) for amino acid identification.

The N-terminal sequence of the purified FTFB was determined and found to be: (A) QVESNNYNGVAEVNTERQANGQI (G) (V) (D) (SEQ ID No.6). Three internal peptide sequences of the purified FTFB were determined: (M) (A) HLDV WDSWPVQDP (V) (SEQ ID No. 7); NAGSIFGT (K) (SEQ ID No. 8); and V (E) (E) VYSPKVSTLMASDEVE (SEQ ID No. 9).

The following primers were designed on the basis of the N-terminal and internal peptide fragments of FTFB. Listed from left to right are: primer name, source peptide fragment and sequence (from 5′ to 3′). FTFB1+FTFB3i yields approximately a 1400 bp product in a PCR reaction. FTFB1 forward (N-terminal): AA T/C-TAT-AA T/C-GG T/C-GTT-GC G/A-T/C GA-AGT (SEQ ID No. 25); and FTFB3i reverse (Internal 3): TAC-CGN-A/T C/G N-CTA-CTT-CAA-CTT (SEQ ID No. 26). The FTFB gene was partly isolated by PCR with primers FTFB1 and FTFB3i. PCR with these primers yielded a 1385 bp amplicon, which after sequencing showed high homology to ftfA and SacB from Streptococcus mutans.

### Example 3

Oxidation of levans

For TEMPO-mediated oxidation, a levan according to the invention prepared as described above (dry weight 1 g, 6.15 mmol of monomeric units present, molar weight 10-1,000 kDa) was resuspended in 100 ml water. Next, 2,2,6,6-tetramethylpiperidine-1-oxyl (TEMPO; 1% by weight compared to the polysaccharide (0.01 g, 0.065 mmol)) was added and resuspended in 20 min. Sodium bromide (0.75 g, 7.3 mmol) was added and the suspension was cooled down to 0° C. This reaction also proceeded without bromide. A solution of hypochlorite (6 ml, 15% solution, 12.6 mmol) was adjusted to pH 10.0 with 3M HCl and cooled to 0C. This solution was added to the suspension of the polysaccharide and TEMPO. The course of the reaction was followed by monitoring the consumption of sodium hydroxide solution, which is equivalent to the formation of uronic acid. After 30 min, 60 ml 0.1M NaOH was consumed. This amount corresponds to the formation of 97% uronic acid. Thereafter, the solution was poured out in 96% ethanol (comprising 70% of the volume of the solution) causing the product to precipitate. The white precipitate was centrifuged, resuspended in ethanol/water (70/30 v/v) and centrifuged again. Next, the precipitate was resuspended in 96% ethanol and centrifuged. The obtained product was dried at reduced pressure. The uronic acid content was determined by means of the uronic acid assay according to Blumenkrantz and Abdoe-Hansen (Anal. Biochem., 54 (1973), 484). A calibration curve was generated using polygalacturonic acid (5, 10, 15 and 20 μg). With this calibration curve the uronic acid content in a sample of 20 μg of the product was determined. The obtained result was a content of 95% uronic acid with a yield of 96%.

Partial Oxidation

For partial oxidation, a levan according to the invention (dry weight 2 g, 12.3 mmol) was resuspended in 25 ml water. Next, TEMPO (1% by weight compared to the polysaccharide (0.02 g, 0.13 mmol)) was added, resuspended in 20 min and cooled to 0° C. A solution of hypochlorite (1 ml, 15% solution, 2.1 mmol) was adjusted to pH 9.0 with 3M HCl and cooled down to 0° C. This solution was added to the suspension of the polysaccharide and TEMPO. Within 5 min the mixture became a solid gel, due to the formation of aldehydes.

### Example 4

Adhesion of Lactobacillus reuteri strains to Caco-2 cell lines

The adhesion of Lactobacillus reuteri strains to Caco-2 cell lines was determined as described below. Firstly, a bacterial suspension was prepared as follows. Lactobacillus reuteri strains LB 121, 35-5, K24 and DSM20016 and L. rhamnosus LGG (a well known probiotic strain with good adhering properties) were cultured in MRS broth supplemented with 5 μl/ml of methyl-1,2-[³H]-thymidine at 37° C. for 18-20 h before the adhesion assays. The cultures were harvested by centrifugation, washed with phosphate buffered saline (PBS) and resuspended in PBS or PBS supplemented with 30 g/l sucrose (see Table 3) to a final density of about 2×10⁹ cfu/ml. Prior to the adhesion assay, the cell suspensions in PBS with 30 g/l sucrose were incubated for 1 hour at 37° C., whereas the cell suspensions in PBS were kept on ice for 1 hour. After incubation at 37° C., the suspensions in PBS with sucrose were centrifuged and the cells were washed with and resuspended in PBS to a final density of about 2×10⁹ cfu/ml.

Caco-2 cells were cultured as follows. Subcultures of Caco-2 cells (ATCC, code HTB 37, human colon adenocarcinoma), stored as frozen stock cultures in liquid nitrogen were used for the adhesion tests. The Caco-2 cells were grown in culture medium consisting of Dulbecco's modified Eagle medium (DMEM), supplemented with heat-inactivated fetal calf serum (10% v/v), non-essential amino acids (1% v/v), L-glutamine (2 mM) and gentamycin (50 μg/ml). About 2,000,000 cells were seeded in 75 cm² tissue culture flasks containing culture medium and cultured in a humidified incubator at 37° C. in air containing 5% CO₂. Near confluent Caco-2 cell cultures were harvested by trypsinisation and resuspended in culture medium. The number of cells was established using a Bürker-Türk counting chamber.

TABLE 3
Incubation of the different Lactobacillus
strains prior to the adhesion assays.

| Lactobacillus | | Polysaccharide | |
|---|---|---|---|
| strain | Extra incubation | produced | Group |
| reuteri 121 | PBS sucrose, 37° C. for | glucan and fructan | As |
| | 1 hr | | |
| reuteri 35-5 | PBS sucrose, 37° C. for | glucan | Bs |
| | 1 hr | | |
| reuteri K24 | PBS sucrose, 37° C. for | none | Cs |
| | 1 hr | | |
| reuteri 121 | PBS on ice | none | D |
| reuteri | PBS on ice | none | E |
| DSM20016* | | | |
| rhamnosus GG | PBS on ice | none | F |

*Type strain of L. reuteri

For the following experiments a Caco-2 monolayer transport system was used. Caco-2 cells cultured in a two-compartment transport system are commonly used to study the intestinal, epithelial permeability. In this system the Caco-2 cell differentiates into polarized columnar cells after reaching confluency. The Caco-2 system has been shown to simulate the passive and active transcellular tranport of electrolytes, sugars, amino acids and lipophilic compounds (Hillgren et al. 1995, Dulfer et al., 1996, Duizer et al., 1997). Also, a clear correlation between the in vivo absorption and the permeability across the monolayers of Caco-2 cells has been reported (Artursson and Karlsson, 1990). For the present transport studies, Caco-2 cells were seeded on semi-permeable filter inserts (12 wells Transwell plates, Costar) at ca. 100,000 cells per filter (growth area±1 cm² containing 2.5 ml culture medium). The cells on the insert were cultured for 17 to 24 days at 37° C. in a humidified incubator containing 5% CO₂ in air. During this culture period the cells have been subjected to an enterocyte-like differentiation. Gentamycin was eliminated from the culture medium two days prior to the adhesion assays.

The adhesion assay was performed as follows. PBS was used as exposure medium. 25 μl of a bacterial suspension (2×10⁹ cfu/ml ) were added to 0.5 ml medium. The apical side of the Caco-2 monolayers was incubated with the bacterial suspensions for 1 hour at 37° C. After incubation, remaining fluid was removed and the cells were washed three times with 1 ml PBS. Subsequently, the Caco-2 monolayers were digested overnight with 1 ml 0.1M NaOH, 1% SDS. The lysate was mixed with 10 ml Hionic Fluor scintillation liquid and the radioactivity was measured by liquid scintillation counting using a LKB/Wallac scintillation counter. As a control, the radioactivity of the bacterial suspensions was measured. For each test group, the percentage of bacteria attached to the monolayers was calculated. All adhesion tests were performed in quadruple. In Table 4 the results of the bacterial adhesion test to Caco-2 cell lines are given. From the results can be concluded that the glucans and the fructans contribute to the adherence of Lactobacillus reuteri to Caco-2 cellines. This could indicate that Lactobacillus reuteri strains producing EPS possess improved probiotic characteristics or that Lactobacillus reuteri and its polysaccharides could function as an excellent symbiotic.

TABLE 4
The results of the bacterial adhesion test to Caco-2 cell lines.

| Group | % of bacteria |
|---|---|
| (see Table | bound to the |
| 1) | monolayer |
| As | 6.5 |
| Bs | 5.7 |
| Cs | 1.8 |
| D | 2.3 |
| E | 0.9 |
| F | 1.3 |

### DESCRIPTION OF THE FIGURES

FIG. 1: SEQ ID No. 1; The deduced amino acid sequence of the novel inulosucrase of Lactobacillus reuteri (amino acid 1-789). The nucleic acid (SEQ ID NO: 4) and deduced amino acid sequence (SEQ ID NOs: 27 and 1) of the novel inulosucrase of Lactobacillus reuteri. Also encompassed within the figure is the comparison peptide (SEQ ID NO: 28). Furthermore, the designations and orientation (<for 3′ to 5′ and >for 5′ to 3′) of the primers and the restriction enzymes used for (inverse) PCR, are shown at the right hand side. Putative start codons (ATG, at positions 41 and 68) and stop codon (TAA, at position 2435) are shown in bold. The positions of the primers used for PCR are shown in bold/underlined. The NheI restriction sites (at positions 1154 and 2592) used for inverse PCR are underlined. The primers used and their exact positions in the inulosucrase sequence are shown in table 1. Starting at amino acid 690, the 20 PXX (residues 690-749 of SEQ IN NO: 1) repeats are underlined. At amino acid 755 the LPXTG (SEQ ID NO: 5) motif is underlined.

FIG. 2: Dendrogram of bacterial and plant fructosyltransferases. The horizontal distances are a measure for the difference at the amino acid sequence level. 10% difference is indicated by the upper bar. Bootstrap values (in percentages) are given at the root of each tree. Fructosyltransferases of Gram positive bacteria are indicated in the lower half of the figure (B. staerothermophilus SurB; B. amyloliquefaciens SacB; B. subtilis SacB; S. mutans SacB; L. reuteri FtfA (inulosucrase); S. salivarius Ftf). Plant fructosyltransferases are indicated in the middle part of the figure (Cynara scolymus Ss-1ft; Allium cepa F-6gft; Hordeum vulgare Sf-6ft). Fructosyltransferases of Gram negative bacteria are shown in the upper part of the figure (Z. mobilis LevU; Z. mobilis SucE2; Z. mobilis SacB; E. amylovora Lcs; A. diazotrophicus LsdA).

FIG. 3: SEQ ID No. 2; The N-terminal (SEQ ID NO: 6) and three internal amino acid sequences (SEQ ID NOs: 7-9) of the novel levansucrase of Lactobacillus reuteri.

FIG. 4: Parts of an alignment of the deduced amino acid sequences of some bacterial fructosyltransferase genes (SEQ ID NOs: 29-40). Sequences in bold indicate the consensus sequences used to construct the degenerated primers 5ftf, 6ftfi and 12 ftfi. (*) indicates a position with a fully conserved amino acid residue. (:) indicates a position with a fully conserved ‘strong’ group: STA, NEQK, NHQK, NDEQ, QHRK, MILV, MILF, HY, FYW. (.) indicates a position with a fully conserved ‘weaker’ group: CSA, ATV, SAG, STNK, STPA, SGND, SNDEQK, NDEQHK, NEQHRK, FVLIM, HFY. Groups are according to the Pam250 residue weight matrix described by Altschul et al. (1990) J. Mol. Biol. 215, 403-410.

FIG. 5: The strategy used for the isolation of the inulosucrase gene from Lactobacillus reuteri 121 chromosomal DNA.

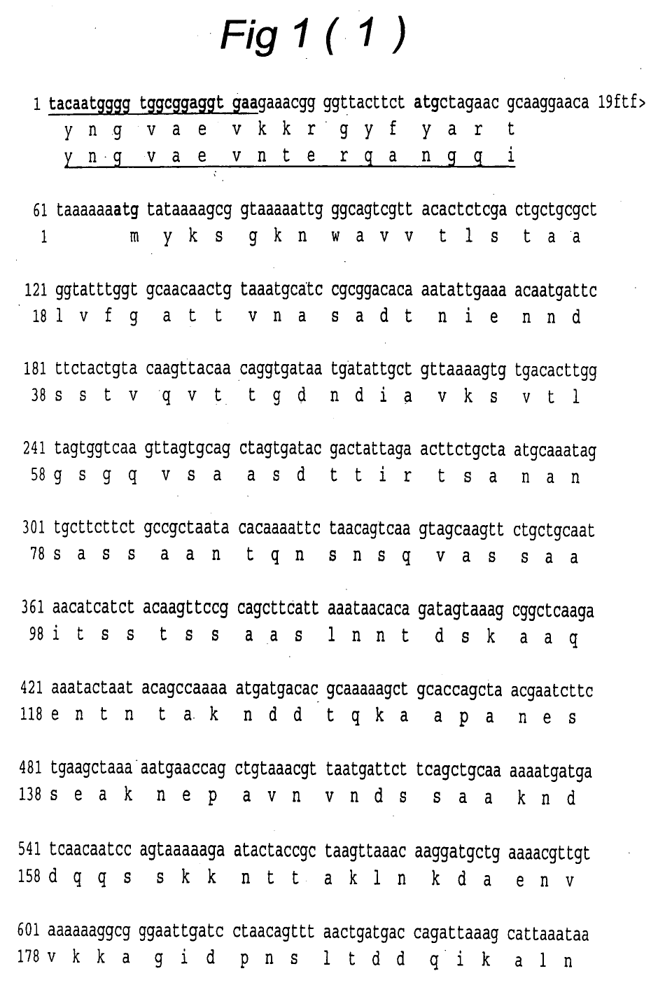

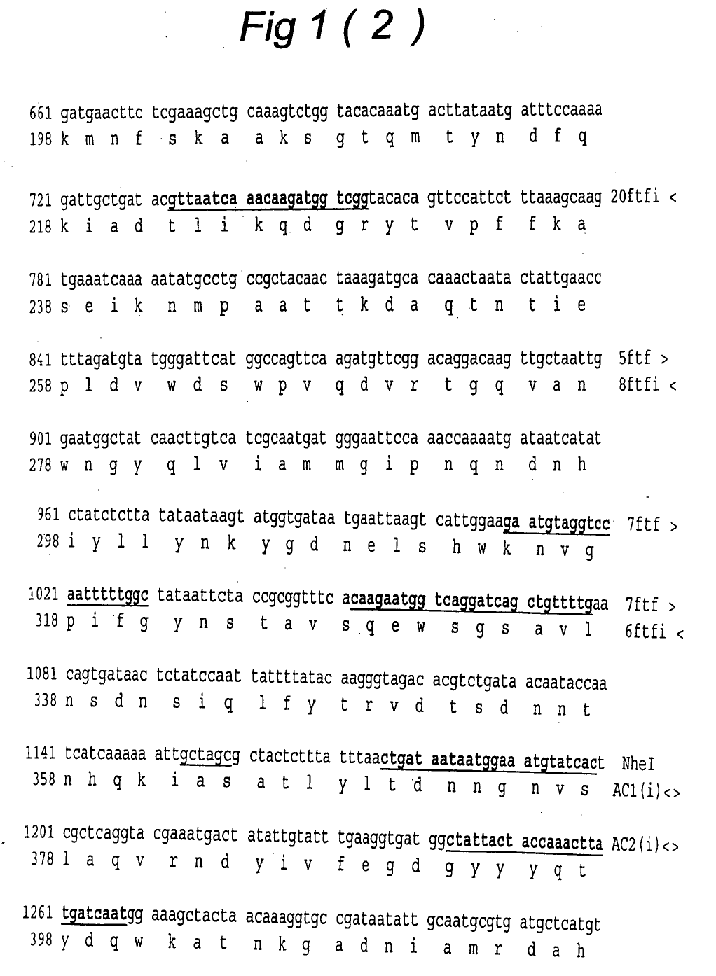

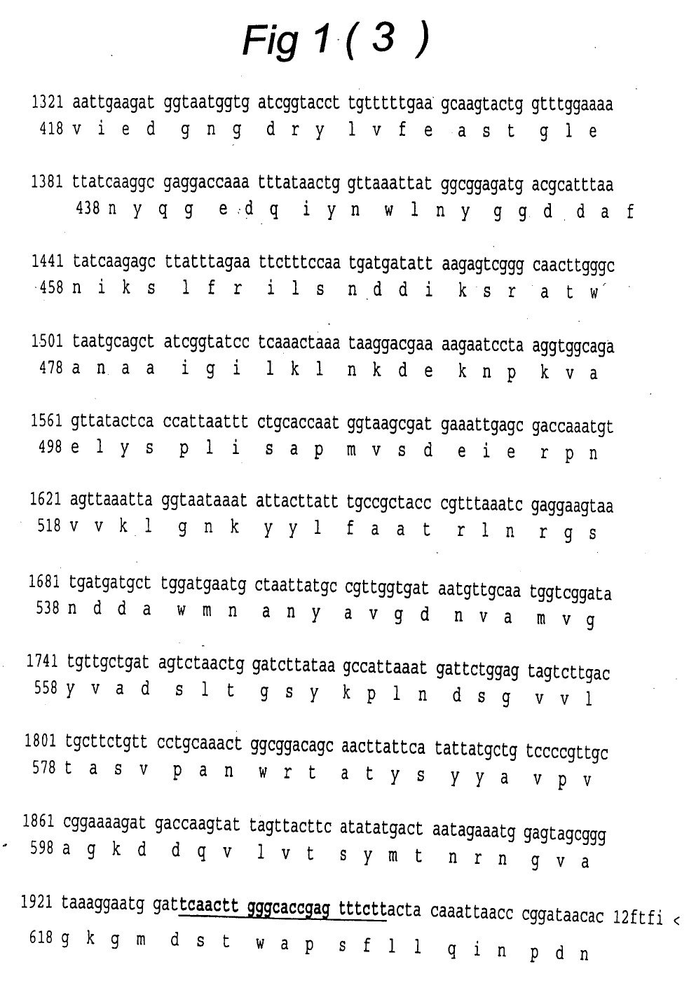

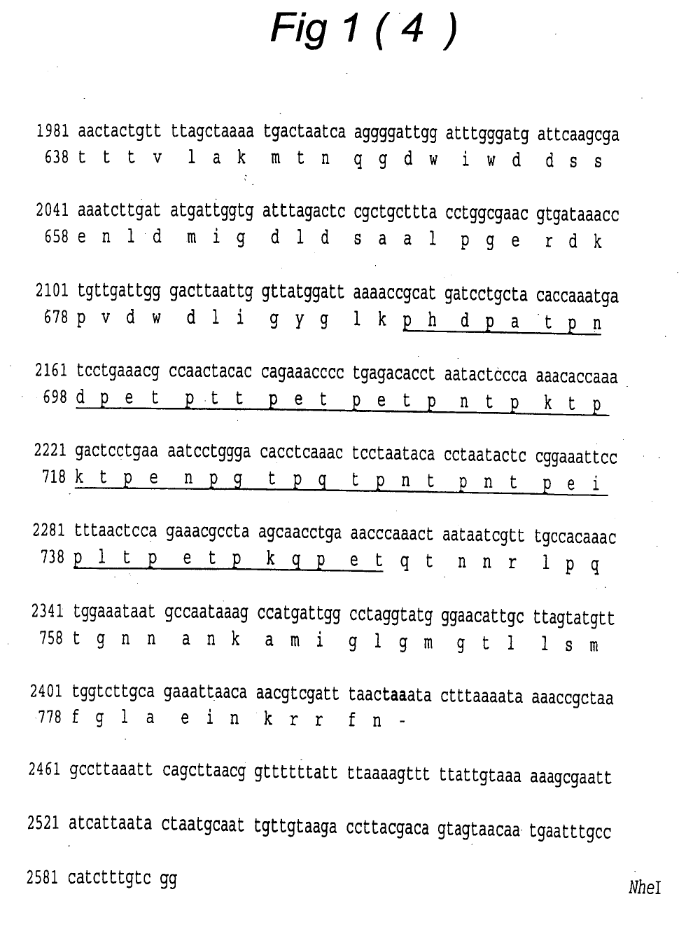

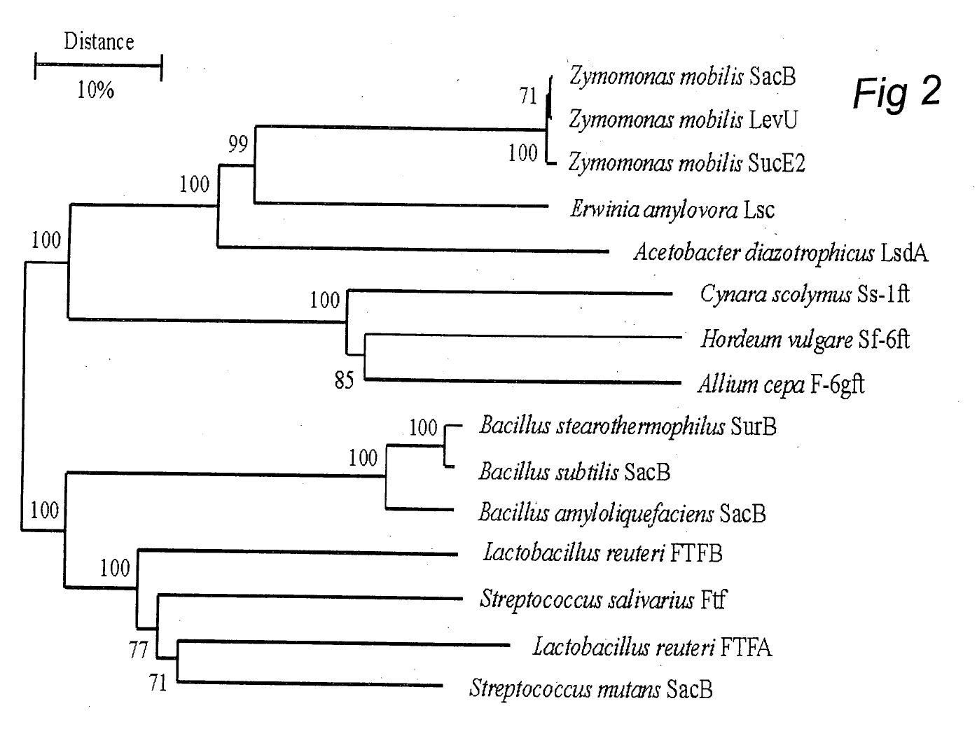

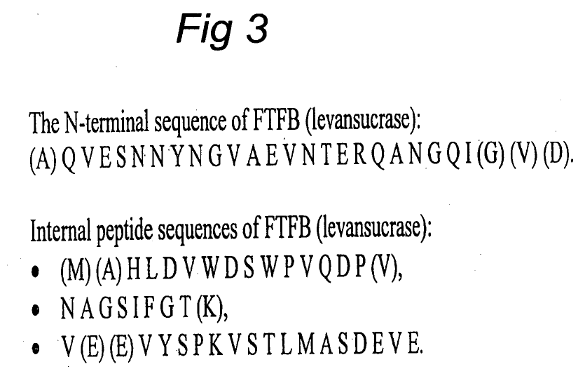

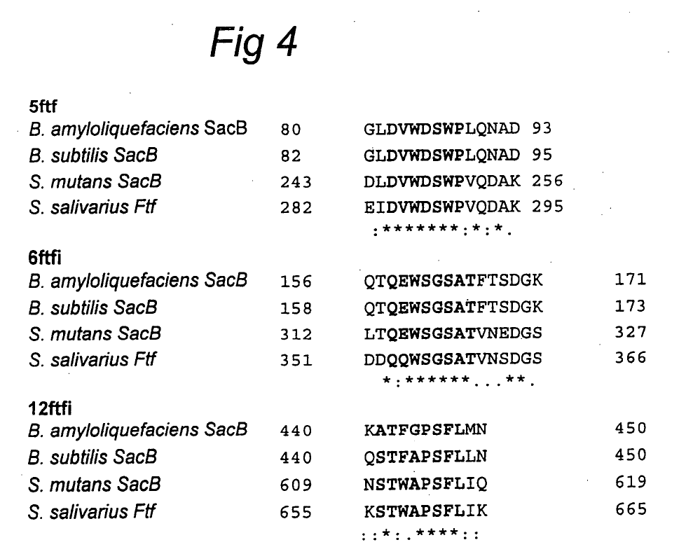

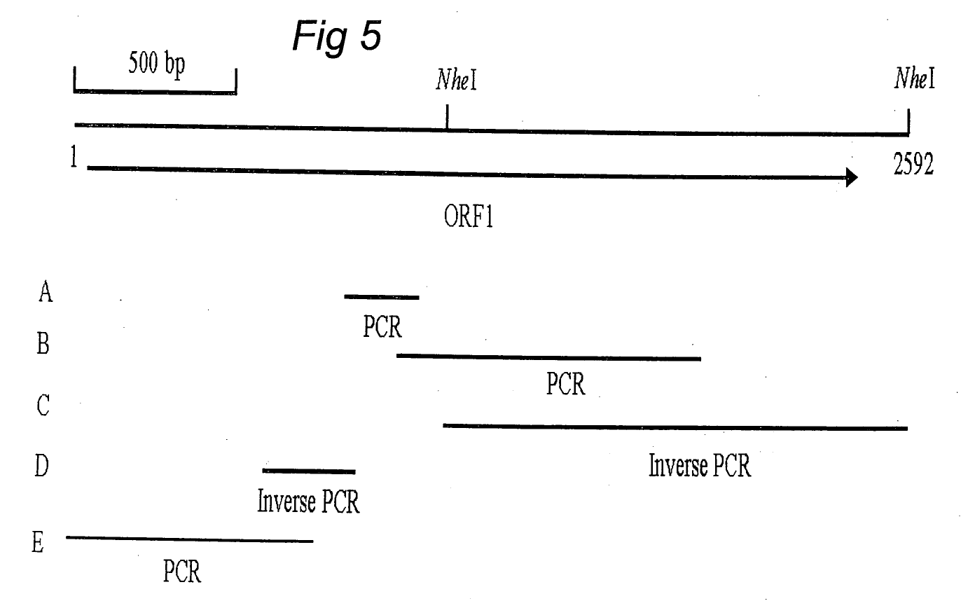

We claim:

1. A process of producing one or more fructans selected from fructans having P(2-1) linked D-fructosyl units and fructans having β(2-6) linked D-fructosyl units, comprising subjecting a fructose source selected from sucrose, raffinose, stachyose and fructo-oligosaccharides to a Lactobacillus strain containing and capable of expressing at least one protein having fructosyltransferase activity, under non-growth conditions, to obtain a mixture containing said one or more fructans.

2. The process of claim 1 for producing from fructans having β(2-1) linked D-fructosyl units, wherein said Lactobacillus strain contains at least one protein having fructosyltransferase activity and exhibiting at least 85% amino acid identity, as determined by the BLAST algorithm, with an amino acid sequence of SEQ ID No. 1.

3. The process of claim 1 for producing from fructans having β(2-6) linked D-fructosyl units, wherein said Lactobacillus strain contains at least one protein having fructosyltransferase activity and exhibiting at least 85% amino acid identity, as determined by the BLAST algorithm, with an amino acid sequence of SEQ ID No. 11.

4. The process according to claim 1, further comprising separating said fructans from said Lactobacillus strain and adding a food or beverage composition to said fructans, to obtain a prebiotic composition.

5. The process according to claim 1, further comprising adding a food or beverage composition to said mixture, to obtain a synbiotic composition.

6. The process according to claim 1, further comprising chemically modifying said one or more fructans by simultaneous 3- and 4-oxidation, by 1- or 6-oxidation, phosphorylation, acylation, alkylation, hydroxyalkylation, carboxymethylation, epoxyalkylation, aminoalkylation of one or more anhydrofructose units of said fructans, or by hydrolysis.

7. A process of producing a chemically modified fructan having at least 100 β(2-1) linked or β(2-6) linked D-fructosyl units, comprising chemically modifying said fructan by simultaneous 3- and 4-oxidation, by 1- or 6-oxidation, phosphorylation, acylation, alkylation, hydroxyalkylation, carboxymethylation, epoxyalkylation, aminoalkylation of one or more anhydrofructose units of said fructans.

8. The process of claim 7, wherein a fructan having β(2-6) linked D-fructosyl units is chemically modified by 1-oxidation using a nitroxyl catalyst.

9. The process of claim 8, wherein partial 1-oxidation of a fructan having β(2-6) linked D-fructosyl units is carried out to obtain a product containing both aldehyde and carboxylic functions.

10. A chemically modified fructan having at least 100 p(2-6) linked D-fructosyl units, containing between 1 and 100 1-aldehyde/carboxyl groups per 100 D-fructosyl units.

11. The modified fructan of claim 10, containing between 1 and 50 1-aldehyde groups and between 1 and 50 1-carboxyl groups per 100 D-fructosyl units.


SEQUENCE LISTING (40 sequences)

SEQ ID NO: 1 - protein, 789 aa, Lactobacillus reuteri
MYKSGKNWAVVTLSTAALVFGATTVNASADTNIENNDSSTVQVTTGDNDIAVKSVTLGSGQVSAASDTTIRTSANANSASSAANTQNSNSQVASSAAITSSTSSAASLNNTDSKAAQENTNTAKNDDTQKAAPANESSEAKNEPAVNVNDSSAAKNDDQQSSKKNTTAKLNKDAENVVKKAGIDPNSLTDDQIKALNKMNFSKAAKSGTQMTYNDFQKIADTLIKQDGRYTVPFFKASEIKNMPAATTKDAQTNTIEPLDVWDSWPVQDVRTGQVANWNGYQLVIAMMGIPNQNDNHIYLLYNKYGDNELSHWKNVGPIFGYNSTAVSQEWSGSAVLNSDNSIQLFYTRVDTSDNNTNHQKIASATLYLTDNNGNVSLAQVRNDYIVFEGDGYYYQTYDQWKATNKGADNIAMRDAHVIEDGNGDRYLVFEASTGLENYQGEDQIYNWLNYGGDDAFNIKSLFRILSNDDIKSRATWANAAIGILKLNKDEKNPKVAELYSPLISAPMVSDEIERPNVVKLGNKYYLFAATRLNRGSNDDAWMNANYAVGDNVAMVGYVADSLTGSYKPLNDSGVVLTASVPANWRTATYSYYAVPVAGKDDQVLVTSYMTNRNGVAGKGMDSTWAPSFLLQINPDNTTTVLAKMTNQGDWIWDDSSENLDMIGDLDSAALPGERDKPVDWDLIGYGLKPHDPATPNDPETPTTPETPETPNTPKTPKTPENPGTPQTPNTPNTPEIPLTPETPKQPETQTNNRLPQTGNNANKAMIGLGMGTLLSMFGLAEINKRRFN

SEQ ID NO: 2 - DNA, 2367 bp, Lactobacillus reuteri
atgtataaaagcggtaaaaattgggcagtcgttacactctcgactgctgcgctggtatttggtgcaacaactgtaaatgcatccgcggacacaaatattgaaaacaatgattcttctactgtacaagttacaacaggtgataatgatattgctgttaaaagtgtgacacttggtagtggtcaagttagtgcagctagtgatacgactattagaacttctgctaatgcaaatagtgcttcttctgccgctaatacacaaaattctaacagtcaagtagcaagttctgctgcaataacatcatctacaagttccgcagcttcattaaataacacagatagtaaagcggctcaagaaaatactaatacagccaaaaatgatgacacgcaaaaagctgcaccagctaacgaatcttctgaagctaaaaatgaaccagctgtaaacgttaatgattcttcagctgcaaaaaatgatgatcaacaatccagtaaaaagaatactaccgctaagttaaacaaggatgctgaaaacgttgtaaaaaaggcgggaattgatcctaacagtttaactgatgaccagattaaagcattaaataagatgaacttctcgaaagctgcaaagtctggtacacaaatgacttataatgatttccaaaagattgctgatacgttaatcaaacaagatggtcggtacacagttccattctttaaagcaagtgaaatcaaaaatatgcctgccgctacaactaaagatgcacaaactaatactattgaacctttagatgtatgggattcatggccagttcaagatgttcggacaggacaagttgctaattggaatggctatcaacttgtcatcgcaatgatgggaattccaaaccaaaatgataatcatatctatctcttatataataagtatggtgataatgaattaagtcattggaagaatgtaggtccaatttttggctataattctaccgcggtttcacaagaatggtcaggatcagctgttttgaacagtgataactctatccaattattttatacaagggtagacacgtctgataacaataccaatcatcaaaaaattgctagcgctactctttatttaactgataataatggaaatgtatcactcgctcaggtacgaaatgactatattgtatttgaaggtgatggctattactaccaaacttatgatcaatggaaagctactaacaaaggtgccgataatattgcaatgcgtgatgctcatgtaattgaagatggtaatggtgatcggtaccttgtttttgaagcaagtactggtttggaaaattatcaaggcgaggaccaaatttataactggttaaattatggcggagatgacgcatttaatatcaagagcttatttagaattctttccaatgatgatattaagagtcgggcaacttgggctaatgcagctatcggtatcctcaaactaaataaggacgaaaagaatcctaaggtggcagagttatactcaccattaatttctgcaccaatggtaagcgatgaaattgagcgaccaaatgtagttaaattaggtaataaatattacttatttgccgctacccgtttaaatcgaggaagtaatgatgatgcttggatgaatgctaattatgccgttggtgataatgttgcaatggtcggatatgttgctgatagtctaactggatcttataagccattaaatgattctggagtagtcttgactgcttctgttcctgcaaactggcggacagcaacttattcatattatgctgtccccgttgccggaaaagatgaccaagtattagttacttcatatatgactaatagaaatggagtagcgggtaaaggaatggattcaacttgggcaccgagtttcttactacaaattaacccggataacacaactactgttttagctaaaatgactaatcaaggggattggatttgggatgattcaagcgaaaatcttgatatgattggtgatttagactccgctgctttacctggcgaacgtgataaacctgttgattgggacttaattggttatggattaaaaccgcatgatcctgctacaccaaatgatcctgaaacgccaactacaccagaaacccctgagacacctaatactcccaaaacaccaaagactcctgaaaatcctgggacacctcaaactcctaatacacctaatactccggaaattcctttaactccagaaacgcctaagcaacctgaaacccaaactaataatcgtttgccacaaactggaaataatgccaataaagccatgattggcctaggtatgggaacattgcttagtatgtttggtcttgcagaaattaacaaacgtcgatttaac

SEQ ID NO: 3 - DNA, 2394 bp, Lactobacillus reuteri
atgctagaacgcaaggaacataaaaaaatgtataaaagcggtaaaaattgggcagtcgttacactctcgactgctgcgctggtatttggtgcaacaactgtaaatgcatccgcggacacaaatattgaaaacaatgattcttctactgtacaagttacaacaggtgataatgatattgctgttaaaagtgtgacacttggtagtggtcaagttagtgcagctagtgatacgactattagaacttctgctaatgcaaatagtgcttcttctgccgctaatacacaaaattctaacagtcaagtagcaagttctgctgcaataacatcatctacaagttccgcagcttcattaaataacacagatagtaaagcggctcaagaaaatactaatacagccaaaaatgatgacacgcaaaaagctgcaccagctaacgaatcttctgaagctaaaaatgaaccagctgtaaacgttaatgattcttcagctgcaaaaaatgatgatcaacaatccagtaaaaagaatactaccgctaagttaaacaaggatgctgaaaacgttgtaaaaaaggcgggaattgatcctaacagtttaactgatgaccagattaaagcattaaataagatgaacttctcgaaagctgcaaagtctggtacacaaatgacttataatgatttccaaaagattgctgatacgttaatcaaacaagatggtcggtacacagttccattctttaaagcaagtgaaatcaaaaatatgcctgccgctacaactaaagatgcacaaactaatactattgaacctttagatgtatgggattcatggccagttcaagatgttcggacaggacaagttgctaattggaatggctatcaacttgtcatcgcaatgatgggaattccaaaccaaaatgataatcatatctatctcttatataataagtatggtgataatgaattaagtcattggaagaatgtaggtccaatttttggctataattctaccgcggtttcacaagaatggtcaggatcagctgttttgaacagtgataactctatccaattattttatacaagggtagacacgtctgataacaataccaatcatcaaaaaattgctagcgctactctttatttaactgataataatggaaatgtatcactcgctcaggtacgaaatgactatattgtatttgaaggtgatggctattactaccaaacttatgatcaatggaaagctactaacaaaggtgccgataatattgcaatgcgtgatgctcatgtaattgaagatggtaatggtgatcggtaccttgtttttgaagcaagtactggtttggaaaattatcaaggcgaggaccaaatttataactggttaaattatggcggagatgacgcatttaatatcaagagcttatttagaattctttccaatgatgatattaagagtcgggcaacttgggctaatgcagctatcggtatcctcaaactaaataaggacgaaaagaatcctaaggtggcagagttatactcaccattaatttctgcaccaatggtaagcgatgaaattgagcgaccaaatgtagttaaattaggtaataaatattacttatttgccgctacccgtttaaatcgaggaagtaatgatgatgcttggatgaatgctaattatgccgttggtgataatgttgcaatggtcggatatgttgctgatagtctaactggatcttataagccattaaatgattctggagtagtcttgactgcttctgttcctgcaaactggcggacagcaacttattcatattatgctgtccccgttgccggaaaagatgaccaagtattagttacttcatatatgactaatagaaatggagtagcgggtaaaggaatggattcaacttgggcaccgagtttcttactacaaattaacccggataacacaactactgttttagctaaaatgactaatcaaggggattggatttgggatgattcaagcgaaaatcttgatatgattggtgatttagactccgctgctttacctggcgaacgtgataaacctgttgattgggacttaattggttatggattaaaaccgcatgatcctgctacaccaaatgatcctgaaacgccaactacaccagaaacccctgagacacctaatactcccaaaacaccaaagactcctgaaaatcctgggacacctcaaactcctaatacacctaatactccggaaattcctttaactccagaaacgcctaagcaacctgaaacccaaactaataatcgtttgccacaaactggaaataatgccaataaagccatgattggcctaggtatgggaacattgcttagtatgtttggtcttgcagaaattaacaaacgtcgatttaac

SEQ ID NO: 4 - DNA, 2592 bp, Lactobacillus reuteri
  CDS (1)..(51)
  CDS (68)..(2434)
tacaatggggtggcggaggtgaagaaacggggttacttctatgctagaacgcaaggaacataaaaaaatgtataaaagcggtaaaaattgggcagtcgttacactctcgactgctgcgctggtatttggtgcaacaactgtaaatgcatccgcggacacaaatattgaaaacaatgattcttctactgtacaagttacaacaggtgataatgatattgctgttaaaagtgtgacacttggtagtggtcaagttagtgcagctagtgatacgactattagaacttctgctaatgcaaatagtgcttcttctgccgctaatacacaaaattctaacagtcaagtagcaagttctgctgcaataacatcatctacaagttccgcagcttcattaaataacacagatagtaaagcggctcaagaaaatactaatacagccaaaaatgatgacacgcaaaaagctgcaccagctaacgaatcttctgaagctaaaaatgaaccagctgtaaacgttaatgattcttcagctgcaaaaaatgatgatcaacaatccagtaaaaagaatactaccgctaagttaaacaaggatgctgaaaacgttgtaaaaaaggcgggaattgatcctaacagtttaactgatgaccagattaaagcattaaataagatgaacttctcgaaagctgcaaagtctggtacacaaatgacttataatgatttccaaaagattgctgatacgttaatcaaacaagatggtcggtacacagttccattctttaaagcaagtgaaatcaaaaatatgcctgccgctacaactaaagatgcacaaactaatactattgaacctttagatgtatgggattcatggccagttcaagatgttcggacaggacaagttgctaattggaatggctatcaacttgtcatcgcaatgatgggaattccaaaccaaaatgataatcatatctatctcttatataataagtatggtgataatgaattaagtcattggaagaatgtaggtccaatttttggctataattctaccgcggtttcacaagaatggtcaggatcagctgttttgaacagtgataactctatccaattattttatacaagggtagacacgtctgataacaataccaatcatcaaaaaattgctagcgctactctttatttaactgataataatggaaatgtatcactcgctcaggtacgaaatgactatattgtatttgaaggtgatggctattactaccaaacttatgatcaatggaaagctactaacaaaggtgccgataatattgcaatgcgtgatgctcatgtaattgaagatggtaatggtgatcggtaccttgtttttgaagcaagtactggtttggaaaattatcaaggcgaggaccaaatttataactggttaaattatggcggagatgacgcatttaatatcaagagcttatttagaattctttccaatgatgatattaagagtcgggcaacttgggctaatgcagctatcggtatcctcaaactaaataaggacgaaaagaatcctaaggtggcagagttatactcaccattaatttctgcaccaatggtaagcgatgaaattgagcgaccaaatgtagttaaattaggtaataaatattacttatttgccgctacccgtttaaatcgaggaagtaatgatgatgcttggatgaatgctaattatgccgttggtgataatgttgcaatggtcggatatgttgctgatagtctaactggatcttataagccattaaatgattctggagtagtcttgactgcttctgttcctgcaaactggcggacagcaacttattcatattatgctgtccccgttgccggaaaagatgaccaagtattagttacttcatatatgactaatagaaatggagtagcgggtaaaggaatggattcaacttgggcaccgagtttcttactacaaattaacccggataacacaactactgttttagctaaaatgactaatcaaggggattggatttgggatgattcaagcgaaaatcttgatatgattggtgatttagactccgctgctttacctggcgaacgtgataaacctgttgattgggacttaattggttatggattaaaaccgcatgatcctgctacaccaaatgatcctgaaacgccaactacaccagaaacccctgagacacctaatactcccaaaacaccaaagactcctgaaaatcctgggacacctcaaactcctaatacacctaatactccggaaattcctttaactccagaaacgcctaagcaacctgaaacccaaactaataatcgtttgccacaaactggaaataatgccaataaagccatgattggcctaggtatgggaacattgcttagtatgtttggtcttgcagaaattaacaaacgtcgatttaactaaatactttaaaataaaaccgctaagccttaaattcagcttaacggttttttattttaaaagtttttattgtaaaaaagcgaattatcattaatactaatgcaattgttgtaagaccttacgacagtagtaacaatgaatttgcccatctttgtcgg

SEQ ID NO: 5 - protein, 5 aa, Lactobacillus reuteri
  MOD_RES (3): Any amino acid
LPXTG

SEQ ID NO: 6 - protein, 27 aa, Lactobacillus reuteri
AQVESNNYNGVAEVNTERQANGQIGVD

SEQ ID NO: 7 - protein, 16 aa, Lactobacillus reuteri
MAHLDVWDSWPVQDPV

SEQ ID NO: 8 - protein, 9 aa, Lactobacillus reuteri
NAGSIFGTK

SEQ ID NO: 9 - protein, 19 aa, Lactobacillus reuteri
VEEVYSPKVSTLMASDEVE

SEQ ID NO: 10 - DNA, 4634 bp, Lactobacillus reuteri
  CDS (1220)..(3598)
  RBS (1205)..(1210)
  modified_base (2702)..(2707): a, c, t, g, other or unknown
gttaacaaagacaaaattttatataattcttcaaattaaatttcccactgtaagaacataaatgggtacctgtttgatgggaataatatatttgtaactaaccggccggcacctctttctaatgtgcctaggatgcataatggatgtaaattactagatggcggtttttatacattaacctcgcaggagagaaaagaagcaattagtaaggatccatatgcagataaatttattaggccttatttaggtgctaaaaatttcattcatggaactgctaggtactgtatttggttaaaggacgcaaacccgaaagatatccatcaatcgccatttatactggatagaatcaataaagtagcggaattcagatcgcagcaaaaaagtaaagatacacaaaaatatgcaaaacggcccatgctaacaacacgacttgcctattatagccacgatgtacatacggatatgctgatagtacctgcaacatcatcgcaacgtagagaatatcttccaattggatatgtttcagaaaagaatattgtgtcttattcactaatgctaatccccaatgctagtaattttaatttcggtattctagaatctaaagttcactatatttggttaaaaaacttttgcggtcggttgaagtccgattatcgttattcaaacactattatttataataatttcccttggccgactgttggtgacaagccaggamcaacaccatctctgacactcgctcaaggtatattaaatactcgcaagctctatccagacagctcactggctgatctttatgatccactaacaatgccragttgaactcgtaaagctcatgaagccaatgataaagctgttcttaaagcatatggattgagccctaaagctactgagcaagaaatcgtagaacatctatttaagatgtatgaaaaactgactaaaggtgaaagataactttgtaaaaccaatattttataaagacagtaaatgttaatttgataaaaacatatatttaataaacaaaagtgatataatcaagtagttctttgtattacaaaatacatttaatatctctcagcattttgcatactgggagattttttattgacaaattgtttgaaagtgcttatgatgaaaccgtgtagaaactaattcaatttgataaacgttagacatttctgaggaggaagtcattttggagtacaaagaacataagaaaatgtataaagtcggcaagaattgggccgttgctacattggtatcagcttcaattttaatgggaggggttgtaaccgctcatgctgatcaagtagaaagtaacaattacaacggtgttgctgaagttaatactgaacgtcaagctaatggtcaaattggcgtagatggaaaaattattagtgctaacagtaatacaaccagtggctcgacaaatcaagaatcatctgctactaacaatactgaaaatgctgttgttaatgaaagcaaaaatactaacaatactgaaaatgctgttgttaatgaaaacaaaaatactaacaatactgaaaatgctgttgttaatgaaaacaaaaatactaacaacacagaaaacgataatagtcaattaaagttaactaataatgaacaaccatcagccgctactcaagcaaacttgaagaagctaaatcctcaagctgctaaggctgttcaaaatgccaagattgatgccggtagtttaacagatgatcaaattaatgaattaaataagattaacttctctaagtctgctgaaaagggtgcaaaattgacctttaaggacttagaggggattggtaatgctattgttaagcaagatccacaatatgctattccttattctaatgctaaggaaatcaagaatatgcctgcaacatacactgtagatgcccaaacaggtaagatggctcatcttgatgtctgggactcttggccagtacaagatcctgtcacaggttatgtatctaattacatgggttatcaactagttattgctatgatgggtattccaaattcgccaactggagataatcatatctatcttctttacaacaagtatggtgataatgacttttctcattggcgcaatgcaggttcaatctttggaactaaagaaacaaatgtgttccaagaatggtcaggttcagctattgtaaatgatgatggtacaattcaactatttttcacctcaaatgatacgtctgattacaagttgaatgatcaacgccttgctaccgcaacattaaaccttaatgttgatgataacggtgtttcaatcaagagtgttgataattatcaagttttgtttgaaggtgatggatttcactaccaaacttatgaacaattcgcaaacggcaaagatcgtgaaaatgatgattactgcttacgtgacccacacgttgttcaattagaaaatggtgatcgttatcttgtattcgaagctaatactgggacagaagattaccaaagtgacgaccaaatttataattgggctaactatggtggcgatgatgccttcaatattaagagttccttcaagcttttgaataataagaaggatcgtgaattggctggtttagctaatggtgcacttggtatcttaaagctcactaacaatcaaagtaagccaaaggttgaagaagtatactcaccattggtatctactttgatggcttgcgatgaggtannnnnnaagcttggtgataagtattatctcttctccgtaactcgtgtaagtcgtggttccgatcgtgaattaaccgctaaggataacacaatcgttggtgataacgttgctatgattggttacgtttccgatagcttaatgggtaagtacaagccattaaataactcaggtgtcgtattaactgcatcagtacctgcaaactggcgtactgctacttattcctactatgcagtacctgtagctggtcatcctgatcaagtattaattacttcttacatgagtaacaaggactttgcttcaggtgaaggaaactatgcaacttgggcaccaagtttcttagtacaaatcaatccagatgacacgacaactgtattagcacgtgcaactaaccaaggtgactgggtgtgggacgactctagtcggaacgataatatgctcggtgttcttaaagaaggtgcagctaacagtgccgccttaccaggtgaatggggtaagccagttgactggagtttgattaacagaagtcctggcttaggcttaaagcctcatcaaccagttcaaccaaagattgatcaacctgatcaacaaccttctggtcaaaacactaagaatgtcacaccaggtaatggtgataagcctgctggtaaggcaactcctgataacactaatattgatccaagtgcacaaccttctggtcaaaacactaatattgatccaagtgcacaamcttctggtcaaaacactaagaatgtcacaccaggtaatgagaaacaaggtaagaataccgatgcaaaacaattaccacaaacaggtaataagtctggtttagcaggactttacgctggttcattacttgccttgtttggattggcagcaattgaaaagcgtcacgcttaatagagtaaaaaaacatcctccactcaagttacaagtaggataatatgtattatttctacgcytagtcaagaggrattactggacatannnnnnnnnnnnntccagttaccaagtggaatatagtattattccacgctagtcaggaggattactgacattattggctacatggccggtagtcctcttttcttttgtgacgaattgtcaaaccaagtgcaacggtttctcaaaaaacacctcatatggggtttcataatttaacacttttcgaggacggcggttcagctgatgttggcagaaactgacgtccttatctgtataatcatcaatattagcccttttaggaaagtattccctaattagsccattggtattttcattgggtcctctttcctctggtgaatagggatctggccaatagatagctactcctaaacgtcctcgaatatcattcaagccaagaaattcacgcccatgatctggagtcaatgaatggacaaattctttaggaatagaccctaagagatcaattaagccctgatatttgaattcggagaaggggagttgtccaacaattgccgttataataccagggttaatacggccctgggcctctacggtaatattgtatttttggctcagatcagtgatagaaacccacagatttagcttgccggtggagtgctgcttgaagtcttcaattacttcgttaccatgtttgattgctaatctgatgtgtcgttgttgtggtgtagtaggcatcataccacctcctcataaaataaggtataacaggaatttcttgtactatatgatccttccaatataataatattaggccgataagaaatgaccagctaccatttcttgatgcttagtgaatataatcggatgatacgtcacccctcaacaatccaatttcacggaggtgagtaatcatgccgagagctaggaatgattggaggaacgaacacggtccatgcggcagtggctatttggattttagccaaagcagcgttactgcttgcaaaagctt

SEQ ID NO: 11 - protein, 792 aa, Lactobacillus reuteri
  MOD_RES (495)..(496): Any amino acid
MYKVGKNWAVATLVSASILMGGVVTAHADQVESNNYNGVAEVNTERQANGQIGVDGKIISANSNTTSGSTNQESSATNNTENAVVNESKNTNNTENAVVNENKNTNNTENAVVNENKNTNNTENDNSQLKLTNNEQPSAATQANLKKLNPQAAKAVQNAKIDAGSLTDDQINELNKINFSKSAEKGAKLTFKDLEGIGNAIVKQDPQYAIPYSNAKEIKNMPATYTVDAQTGKMAHLDVWDSWPVQDPVTGYVSNYMGYQLVIAMMGIPNSPTGDNHIYLLYNKYGDNDFSHWRNAGSIFGTKETNVFQEWSGSAIVNDDGTIQLFFTSNDTSDYKLNDQRLATATLNLNVDDNGVSIKSVDNYQVLFEGDGFHYQTYEQFANGKDRENDDYCLRDPHVVQLENGDRYLVFEANTGTEDYQSDDQIYNWANYGGDDAFNIKSSFKLLNNKKDRELAGLANGALGILKLTNNQSKPKVEEVYSPLVSTLMACDEVXXKLGDKYYLFSVTRVSRGSDRELTAKDNTIVGDNVAMIGYVSDSLMGKYKPLNNSGVVLTASVPANWRTATYSYYAVPVAGHPDQVLITSYMSNKDFASGEGNYATWAPSFLVQINPDDTTTVLARATNQGDWVWDDSSRNDNMLGVLKEGAANSAALPGEWGKPVDWSLINRSPGLGLKPHQPVQPKIDQPDQQPSGQNTKNVTPGNGDKPAGKATPDNTNIDPSAQPSGQNTNIDPSAQXSGQNTKNVTPGNEKQGKNTDAKQLPQTGNKSGLAGLYAGSLLALFGLAAIEKRHA

SEQ ID NO: 12 - DNA, 24 bp, Artificial Sequence
  Description of Artificial Sequence Primer
ctgataataatggaaatgtatcac

SEQ ID NO: 13 - DNA, 26 bp, Artificial Sequence
  Description of Artificial Sequence Primer
catgatcataagtttggtagtaatag

SEQ ID NO: 14 - DNA, 24 bp, Artificial Sequence
  Description of Artificial Sequence Primer
gtgatacatttccattattatcag

SEQ ID NO: 15 - DNA, 26 bp, Artificial Sequence
  Description of Artificial Sequence Primer
ctattactaccaaacttatgatcatg

SEQ ID NO: 16 - DNA, 38 bp, Artificial Sequence
  Description of Artificial Sequence Primer
ccatggccatggtagaacgcaaggaacataaaaaaatg

SEQ ID NO: 17 - DNA, 38 bp, Artificial Sequence
  Description of Artificial Sequence Primer
agatctagatctgttaaatcgacgtttgttaatttctg

SEQ ID NO: 18 - DNA, 21 bp, Artificial Sequence
  Description of Artificial Sequence Primer
gaygtntgggaywsntgggcc

SEQ ID NO: 19 - DNA, 23 bp, Artificial Sequence
  modified_base (3): a, c, t, g, other or unknown
gtngcnswnccnswccaytsytg

SEQ ID NO: 20 - DNA, 22 bp, Artificial Sequence
  Description of Artificial Sequence Primer
gaatgtaggtccaatttttggc

SEQ ID NO: 21 - DNA, 22 bp, Artificial Sequence
  Description of Artificial Sequence Primer
cctgtccgaacatcttgaactg

SEQ ID NO: 22 - DNA, 23 bp, Artificial Sequence
  Description of Artificial Sequence Primer
arraanswnggngcvmangtnsw

SEQ ID NO: 23 - DNA, 23 bp, Artificial Sequence
  Description of Artificial Sequence Primer
tayaayggngtngcngargtnaa

SEQ ID NO: 24 - DNA, 22 bp, Artificial Sequence
  Description of Artificial Sequence Primer
ccgaccatcttgtttgattaac

SEQ ID NO: 25 - DNA, 24 bp, Artificial Sequence
  Description of Artificial Sequence Primer
aaytataayggygttgcrygaagt

SEQ ID NO: 26 - DNA, 21 bp, Artificial Sequence
  Description of Artificial Sequence Primer
taccgnwsnctacttcaactt

SEQ ID NO: 27 - protein, 17 aa, Lactobacillus reuteri
YNGVAEVKKRGYFYART

SEQ ID NO: 28 - protein, 17 aa, Lactobacillus reuteri
YNGVAEVNTERQANGGI

SEQ ID NO: 29 - protein, 14 aa, Bacillus amyloliquefaciens
GLDVWDSWPLQNAD

SEQ ID NO: 30 - protein, 14 aa, Bacillus subtilis
GLDVWDSWPLQNAD

SEQ ID NO: 31 - protein, 14 aa, Streptococcus mutans
DLDVWDSWPVQDAK

SEQ ID NO: 32 - protein, 14 aa, Streptococcus salivarius
EIDVWDSWPVQDAK

SEQ ID NO: 33 - protein, 16 aa, Bacillus amyloliquefaciens
QTQEWSGSATFTSDGK

SEQ ID NO: 34 - protein, 16 aa, Bacillus subtilis
QTQEWSGSATFTSDGK

SEQ ID NO: 35 - protein, 16 aa, Streptococcus mutans
LTQEWSGSATVNEDGS

SEQ ID NO: 36 - protein, 16 aa, Streptococcus salivarius
DDQQWSGSATVNSDGS

SEQ ID NO: 37 - protein, 11 aa, Bacillus amyloliquefaciens
KATFGPSFLMN

SEQ ID NO: 38 - protein, 11 aa, Bacillus subtilis
QSTFAPSFLLN

SEQ ID NO: 39 - protein, 11 aa, Streptococcus mutans
NSTWAPSFLIQ

SEQ ID NO: 40 - protein, 11 aa, Streptococcus salivarius
KSTWAPSFLIK